(19) United States
(12) Patent Application Publication
(10) Pub. No.: US 2008/0152593 A1
(43) Pub. Date: Jun. 26, 2008

(54) Contrast Agent

(75) Inventors: Sumio Iijima, Nagoya-shi (JP); Jin Miyawaki, Tsukuba-shi (JP); Masako Yudasaka, Tsukuba-shi (JP); Eiichi Nakamura, Bunkyo-ko (JP); Hiroyuki Isobe, Arakawa-ku (JP); Hideki Yorimitsu, Muko-shi (JP); Hideto Imai, Tsukuba-shi (JP)

Correspondence Address: WENDEROTH, LIND & PONACK, L.L.P., 2033 K STREET N. W., SUITE 800, WASHINGTON, DC, 20006-1021, US

(73) Assignees: Japan Science And Technology Agency, Saitama (JP); NEC Corporation, Tokyo (JP)

(21) Appl. No.: 11/793,655

(22) PCT Filed: Dec. 21, 2005
(86) PCT No.: PCT/JP2005/024003

§ 371 Date: Sep. 13, 2007

(30) Foreign Application Priority Data
Dec. 24, 2004 (JP) 2004-374878

(51) Int. Cl. A61K 49/06

(52) U.S. Cl. 424/9.36; 424/9.3

(57) ABSTRACT

A contrast agent characterized in that each of carbon nanohorns forming a carbon nanohorn aggregate has an opening at the side wall or tip, wherein a metal M (at least one metal selected from among paramagnetic metals, ferromagnetic metals, and superparamagnetic metals) or a compound of the metal M is incorporated in or dispersed on each of the carbon nanohorns. A contrast agent characterized in that it contains a Gd oxide. There is provided a contrast agent, which can be mass-produced easily, and satisfies the requirement of low toxicity and enables microscopic diagnoses when used for MRI. A contrast agent characterized in that is contains a carbon nanohorn aggregate.

### TECHNICAL FIELD

The present invention relates to a contrast agent. More specifically, the present invention relates to a novel contrast agent that can be suitably used for MRI diagnoses utilizing nanotechnologies, detection of externally invisible defects in materials and structures, etc.

### BACKGROUND ART

Contrast agents utilizing gadolinium (Gd) have conventionally been used for MRI diagnoses, where Gd is in the state of an ion or an ionic compound. Also, contrast agents utilizing iron (Fe) as an ion or an iron oxide have been used for MRI diagnoses.

Further, with rapid development of nanotechnologies, also MRI contrast agents utilizing the carbon nanostructures fullerenes have recently been proposed. For example, a contrast agent, which contains an anion radical salt of a fullerene as an active ingredient, is proposed in Patent Document 1.

A contrast agent proposed in Patent Document 2 is such that a metal-containing fullerene having an average particle diameter of 0.5 to 1 nm is used as a core, and the surface of the core is covered with a polysaccharide having a functional group selected from the group consisting of sulfone groups, ketone groups, amino groups, and alkyl groups.

Further, in the contrast agent produced in Patent Document 3, one or two Gd atoms are incorporated in a fullerene.

Patent Document 1: JP-A-7-233093

Patent Document 2: JP-A-8-143478

Patent Document 3: JP-A-2001-114713

Patent Document 4: JP-A-2003-20215

Non-Patent Document 1: Hashimoto, A.; Yorimitsu, H.; Ajima, K.; Suenaga, K.; Isobe, H.; Miyawaki, J.; Yudasaka, M.; Iijima, S.; and Nakamura, E., Proc. Natl. Acad. Sci. USA, 101, p. 8527-8530, 2004

### DISCLOSURE OF THE INVENTION

However, in a case where in MRI diagnoses Gd is used as an ion or in an ionic compound such as DTPA (diethylenetriamine pentaacetic acid), EDTA (ethylenediamine tetraacetic acid), or DOTA (tetraazacyclodocedacane-N,N,N,N-tetraacetic acid), there is a fear that the Gd will exhibit toxicity to living bodies, and there is a practicality problem that the Gd is dissolved and diffused in living bodies due to its water solubility. Further, also in the case of Fe, which is usually used as an ion, there is a practicality problem in that it is difficult to prevent the diffusion of the Fe in living bodies.

On the other hand, in the case of using fullerenes as contrast agents for MRI, there is a problem that their sizes are range from 0.5 to 1 nm so that microscopic diagnoses can be carried out, but they cannot enclose large molecules. Further, the fullerenes are disadvantageous in that they are hard to synthesize.

Accordingly, an object of the present invention is, under such circumstances, to provide a contrast agent that satisfies the requirement of low toxicity, enables microscopic diagnoses, and can be easily synthesized.

Further, another object of the invention is to provide a contrast agent that can be used for detection of externally invisible, microscopic defects in materials and structures, etc.

According to a first aspect of the invention, there is provided a contrast agent characterized by comprising a carbon nanohorn aggregate in order to solve the above problems.

According to a second aspect of the invention, there is provided the contrast agent according to the first aspect, wherein each of carbon nanohorns forming the carbon nanohorn aggregate has an opening at the side wall or tip.

According to a third aspect of the invention, there is provided the contrast agent according to the first or second aspect, wherein a metal M or a compound of the metal M is incorporated in each of the carbon nanohorns forming the carbon nanohorn aggregate, wherein M is at least one metal selected from the group consisting of paramagnetic metals, ferromagnetic metals, and superparamagnetic metals.

According to a fourth aspect of the invention, there is provided the contrast agent according to the first or second aspect, wherein a metal M or a compound of the metal M is dispersed on a surface of each of the carbon nanohorns forming the carbon nanohorn aggregate, wherein M is at least one selected from paramagnetic metals, ferromagnetic metals, and superparamagnetic metals.

According to a fifth aspect of the invention, there is provided the contrast agent according to the third or fourth aspect, wherein the metal M or the compound of the metal M has an average particle diameter of 0.3 to 100 nm.

According to a sixth aspect of the invention, there is provided the contrast agent according to any one of the third to fifth aspects, wherein the compound of the metal M is a metal oxide.

According to a seventh aspect of the invention, there is provided the contrast agent according to any one of the third to fifth aspects, wherein the metal M is Gd or Fe.

According to an eighth aspect of the invention, there is provided the contrast agent according to the sixth aspect, wherein the metal oxide is a Gd oxide or an Fe oxide.

### BRIEF DESCRIPTION OF THE DRAWINGS

FIGS. 1(a) to 1(c) are TEM images of contrast agents of gadolinium oxide-containing carbon nanohorn aggregates produced in Example 1, FIGS. 1(a) and 1(b) being TEM images of HT600-Gdox580 and FIG. 1(c) being a TEM image of HT700-Gdox580.

FIG. 2 is a diagram showing the relation between the T1 relaxation time and Gd concentration of each gadolinium oxide-containing carbon nanohorn aggregate produced in Example 1.

FIG. 3 is an MRI photograph of a character-shaped gel obtained by dissolving the gadolinium oxide-containing carbon nanohorn aggregate produced in Example 1 in an agar, which is embedded in an agar gel containing no carbon nanohorn aggregates.

FIG. 4 is a T1 weighted MRI photograph of a character-shaped gel obtained by dissolving an iron oxide-containing carbon nanohorn aggregate produced in Example 2 in an agar, which is embedded in an agar gel containing no carbon nanohorn aggregates.

FIG. 5 is a T2 weighted MRI photograph of the same gel as FIG. 4.

FIG. 6 is a T2 weighted MRI photograph of a carbon nanohorn aggregate produced in Example 3 dissolved in an agar, then formed into a tulip-shaped gel, a cherry blossom-shaped gel, and a “2004” character-shaped gel, and then embedded in an agar gel containing no carbon nanohorn aggregates.

### BEST MODE FOR CARRYING OUT THE INVENTION

Embodiments of the present invention with above features will be described below.

A first contrast agent of the invention is characterized by comprising a carbon nanohorn aggregate.

The carbon nanohorn aggregate used in the first contrast agent may be produced by a method described in Patent Document 4, which the present inventors were involved with. Thus, such a carbon nanohorn aggregate that carbon nanohorns are aggregated with their tips outwardly projecting can be obtained by evaporating a solid carbon elemental substance so that carbon vapor is released into a surrounding gas under a pressure controlled depending on the type of the atmosphere gas. The surrounding gas may be a single gas or a mixed gas of unreactive gases such as N₂ (nitrogen) gas and noble gases (Ar (argon), He (helium), etc.) The solid carbon elemental substance may be evaporated by means of laser, electric arc, etc.

In the carbon nanohorn aggregate used for the first contrast agent, each of the carbon nanohorns forming the aggregate has a diameter of approximately 1 to 10 nm. The carbon nanohorn aggregate may have a shape of “dahlia”, “bud”, etc.

The carbon nanohorn aggregate has a larger diameter and a larger surface area than fullerenes, and has a lower harmfulness to human body than carbon nanotubes.

Further, the carbon nanohorn aggregate surface may be chemically modified depending on the target living body. Of course the contrast agent composed of the carbon nanohorn aggregate can be used for detection of externally invisible, microscopic defects in materials and structures, etc. in addition to MRI diagnoses.

Then a second contrast agent of the invention will be described. The second contrast agent is characterized in that each of the carbon nanohorns forming the carbon nanohorn aggregate has an opening at the side wall or tip. Further, the second contrast agent is such that a metal M (in which M is at least one metal selected from among paramagnetic metals, ferromagnetic metals, and superparamagnetic metals) or a compound of the metal M is incorporated in each of the carbon nanohorns forming the carbon nanohorn aggregate, or such that a metal M (in which M is at least one selected from paramagnetic metals, ferromagnetic metals, and superparamagnetic metals) or a compound of the metal M is dispersed on a surface of each of the carbon nanohorns forming the carbon nanohorn aggregate. However, the metal or the metal compound does not have to be incorporated in or dispersed on all the carbon nanohorns forming the carbon nanohorn aggregate, as long as a required contrast effect is achieved.

In the carbon nanohorn aggregate used for the second contrast agent, the opening has to be formed at the side wall or tip of each carbon nanohorn. In a case where the metal or the metal compound is incorporated in or dispersed on the carbon nanohorn, the incorporation or dispersion is carried out by utilizing the opening. For example, the formation of the opening and the incorporation or dispersion of the metal or the metal compound may be achieved by a method described in Non-Patent Document 1, which the inventors were involved with.

In this method, a heat treatment is carried out in an oxygen atmosphere to form the opening at the side wall or tip of the carbon nanohorn forming the carbon nanohorn aggregate. As described in Non-Patent Document 1, the opening can be formed by a heat treatment at 420° C. and 580° C. in an oxygen atmosphere. The size of the opening can be controlled to be approximately 0.2 to 5 nm by changing the temperature and heating time in the heat treatment, though the size is not limited thereto.

The metal M incorporated or dispersed in the carbon nanohorn may be a paramagnetic metal, a ferromagnetic metal, or a superparamagnetic metal.

The paramagnetic metal may be the elemental substance or an alloy of a rare-earth metal selected from Gd, Ce, Pr, Sm, Eu, Tb, Dy, Er, Ho, Tm, and Yb, or a metal selected from Mn, Ni, Co, Ru, Rh, and Pd. Gd is particularly preferred from the viewpoint of the contrast effect.

The ferromagnetic metal may be the elemental substance or an alloy of Fe, Ni, or Co. Fe is particularly preferred from the viewpoint of the contrast effect.

The superparamagnetic metal may be a fine particle of a ferromagnetic or ferrimagnetic material composed of the elemental substance or an alloy of Fe, Mn, Ni, Co, or Ru. Ferrimagnetic fine particles of Fe oxide are particularly preferred from the viewpoint of the contrast effect.

The compound of the metal M may be an oxide, a carbide, a chloride, etc. Among them, the oxide is preferred from the viewpoint of stability. In the case of dispersing the compound of the metal M, the compound needs to have water resistance, acid resistance, and low toxicity. Thus, the oxide is particularly preferred in this case.

The oxide of the metal M may be an oxide of the above ones, and a Gd oxide such as Gd₂O₃ (GdOₓ, hereinafter referred also to as a gadolinium oxide) is particularly preferably used as a paramagnetic metal oxide. As described above, in a case where Gd is used in the state of an ion or an ionic compound, there is a fear that the Gd has toxicity in living bodies, and there is a practicality problem that the Gd is dissolved and diffused in living bodies due to its water solubility. However, it has been found that the Gd oxide can be effectively used in the contrast agent without such problems. This knowledge was first confirmed by the inventors.

Further, Fe oxide such as Fe₃O₄ (FeOₓ, hereinafter referred also to as a iron oxide) is preferably used as a ferromagnetic or superparamagnetic metal oxide.

It is preferred in view of achieving a sufficient contrast effect that the metal M or the compound of the metal M is a fine particle having a size of about 0.3 to 20 nm in the carbon nanohorn. In particular, the superparamagnetic metal exhibits that property when micronized into a nanometer size.

For example, the metal M or the compound of the metal M may be incorporated in or dispersed on the carbon nanohorn by a method described in Non-Patent Document 1. In this method, for example in the case of incorporating a gadolinium oxide, gadolinium acetate tetrahydrate and the carbon nanohorn aggregate having the opening are mixed and stirred in ethanol, filtrated, dispersed further in ethanol, subjected to an ultrasonication, and filtrated. Then, the resultant is dried and heat-treated in argon gas to obtain the desired product. In the case of incorporating or dispersing an iron oxide, iron acetate is used instead of the gadolinium acetate tetrahydrate. The incorporation and dispersion of the metal M or the compound of the metal M can be selected by controlling the size of the opening. Further, the size of the metal M or the compound of the metal M can be controlled by changing the temperature and time of the heat treatment in an inert gas such as Ar or He.

The invention will be described in more detail below with reference to Examples. It is apparent that the invention is not limited to the above embodiments and following examples, and various changes and modifications can be made in the details.

### EXAMPLES

### Example 1

A graphite material was irradiated in a chamber with a CO₂ laser at the room temperature in an argon gas atmosphere under a pressure of 101 kPa. The graphite material was evaporated to produce a dahlia-shaped carbon nanohorn aggregate. Each carbon nanohorn in the aggregate had a single-layer structure.

Then, to form an opening in the side wall of each carbon nanohorn, the obtained carbon nanohorn aggregate was heat-treated and thus partially-oxidized at 580° C. for 10 minutes under a pressure of 101 kPa while supplying oxygen at a flow rate of 200 cm³/minute.

50 mg of the oxidized carbon nanohorn aggregate (referred to here as NHox) and 50 mg of a gadolinium acetate tetrahydrate Gd(OAc)₃.4H₂O (available from Sigma-Aldrich Japan K.K., purity 99.9% or more) were mixed in 20 cm³ of ethanol in a conical flask, stirred at the room temperature for 24 hours, and filtrated with a membrane filter having a pore diameter of 0.2 μm. The powder obtained by the filtration was dispersed in ethanol (20 cm³), treated with ultrasonic for 20 seconds, and filtrated again. Then, the resultant was dried for 12 hours under vacuum (1 kPa), to obtain a gadolinium acetate-containing carbon nanohorn aggregate (referred to here as GdOAc@NH).

Two samples of the gadolinium acetate-containing carbon nanohorn aggregate were heat-treated at 600° C. for 60 minutes or at 700° C. for 60 minutes respectively under an argon gas atmosphere (pressure 101 kPa, flow rate 300 cm³/minute), to obtain gadolinium oxide-containing carbon nanohorn aggregates (referred to here as HT600-Gdox580 and HT700-Gdox580 respectively). The average particle diameter of the gadolinium acetate in the former aggregate was approximately 5 nm, and that in the latter aggregate was approximately 10 nm.

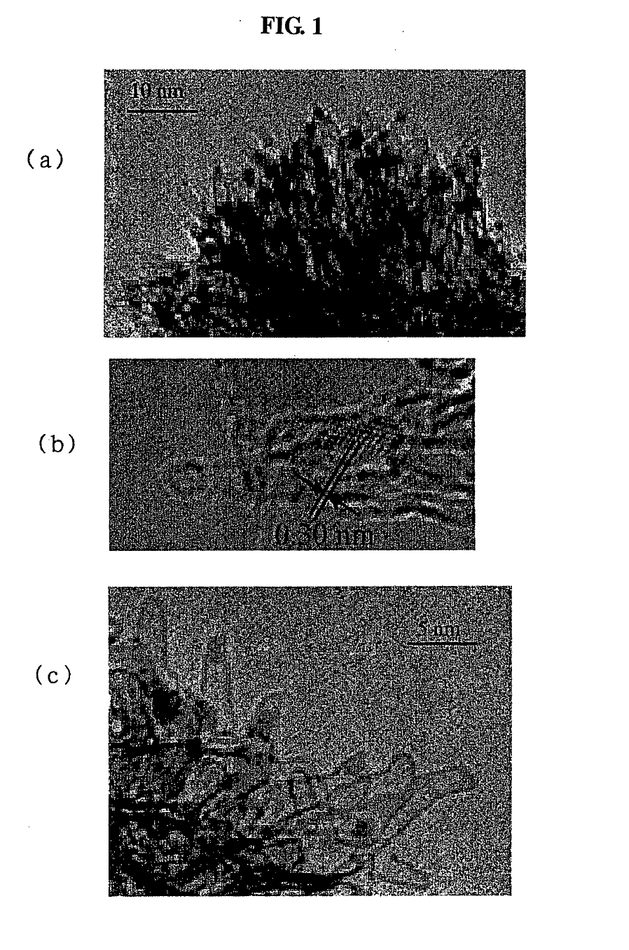

Transmission electron microscope (TEM) images of the HT600-Gdox580 are shown in FIGS. 1(a) and 1(b), and a TEM image of the HT700-Gdox580 is shown in FIG. 1(c). The particles of the gadolinium oxide (Gd₂O₃) are found as black points. It is clear that the gadolinium oxide (Gd₂O₃) particles were incorporated inside the sheaths of the carbon nanohorns. It is also clear that the openings were formed in the side surfaces.

The above produced gadolinium oxide-containing carbon nanohorn aggregates were dispersed in an agar gel, and the T1 relaxation times of the aggregates were measured by NMR.

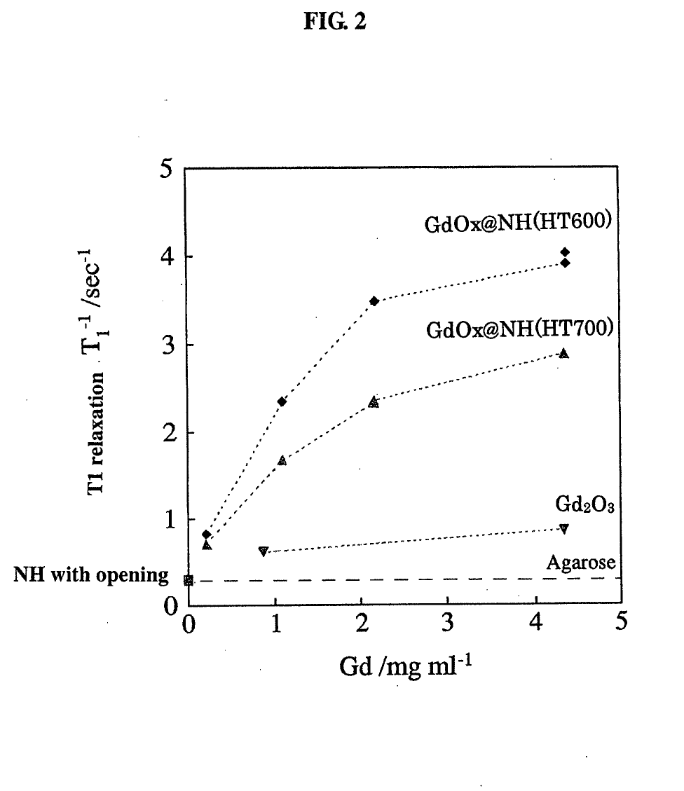

The relation between the measured T1 relaxation time and the Gd concentration (the amount not of Gd₂O₃ but of Gd) of each aggregate is shown in FIG. 2. In FIG. 2, the T1 relaxation (the inverse number of T1) is on the ordinate axis, and as the value thereof is increased, the proton relaxation is accelerated. In FIG. 2, the line of Gd₂O₃ represents data of a commercially available gadolinium oxide (average particle diameter about 100 nm), and the broken line represents data of only the agar gel.

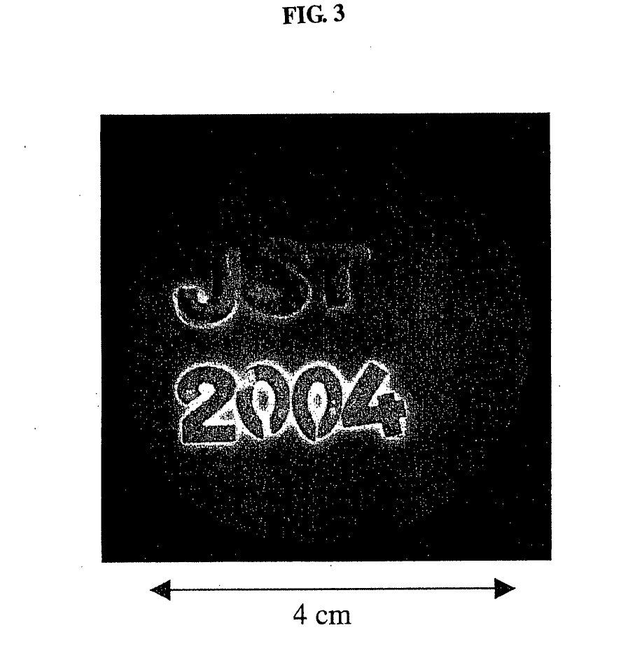

Further, the produced gadolinium oxide-containing carbon nanohorn aggregate was dissolved in an agar, and formed into a “JST2004” character-shaped gel. The gel was embedded in an agar gel containing no carbon nanohorn aggregates, and subjected to MRI. The obtained photograph image is shown in FIG. 3.

As a result, it was confirmed that the gadolinium oxide-containing carbon nanohorn aggregate is effective as a contrast agent.

### Example 2

A dahlia-shaped carbon nanohorn aggregate was produced in the same manner as Example 1.

Then, to form an opening in the side wall of each carbon nanohorn, the obtained carbon nanohorn aggregate was heat-treated and thus partially-oxidized at 580° C. for 10 minutes under a pressure of 101 kPa while supplying oxygen at a flow rate of 200 cm³/minute.

50 mg of the oxidized carbon nanohorn aggregate (referred to here as NHox) and 50 mg of an iron acetate (available from Sigma-Aldrich Japan K.K., purity 99.995% or more) were mixed in 20 cm³ of ethanol in a conical flask, stirred at the room temperature for 24 hours, and filtrated with a membrane filter having a pore diameter of 0.2 μm. The powder obtained by the filtration was dispersed in ethanol (20 cm³), treated with ultrasonic wave for 20 seconds, and filtrated again. Then, the resultant was dried for 12 hours under vacuum (1 kPa), to obtain an iron acetate (Fe(OAc)₂)-containing carbon nanohorn aggregate (referred to as FeOAc@NH).

Two samples of the iron acetate-containing carbon nanohorn aggregate were heat-treated at 400° C. for 60 minutes under an argon gas atmosphere (pressure 101 kPa, flow rate 300 cm³/minute), to obtain iron oxide-containing carbon nanohorn aggregates.

As a result of observing a transmission electron microscope (TEM) image of the iron oxide-containing carbon nanohorn aggregate, the iron oxide particles appeared as black points as in the case of Example 1. Further, it was confirmed that the iron oxide particles are incorporated inside the sheaths of the carbon nanohorns. It was also confirmed that the openings are formed in the side surfaces.

The above produced iron oxide-containing carbon nanohorn aggregates were dispersed in agar gel, and the T1 relaxation times of the aggregates were measured by NMR in the same manner as Example 1. As a result, the relation between the measured T1 relaxation time and the Fe concentration (the amount of not Fe₃O₄ but Fe) was equal to that of Example 1.

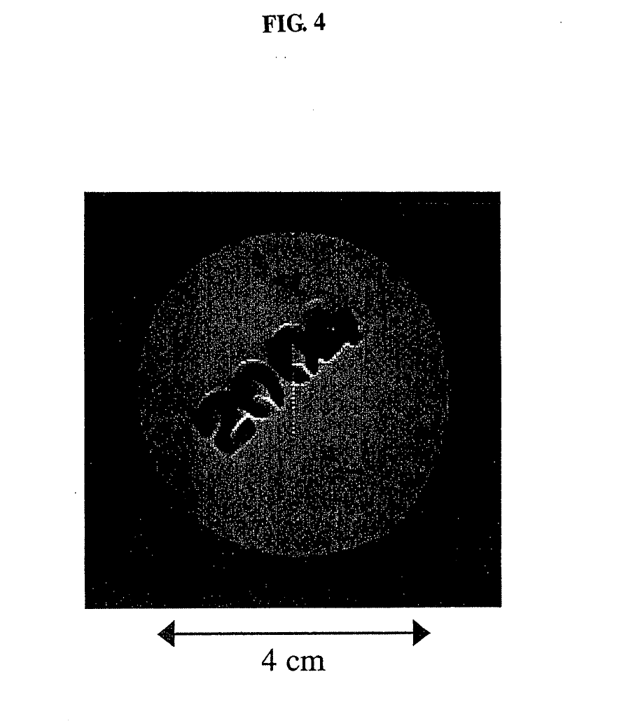

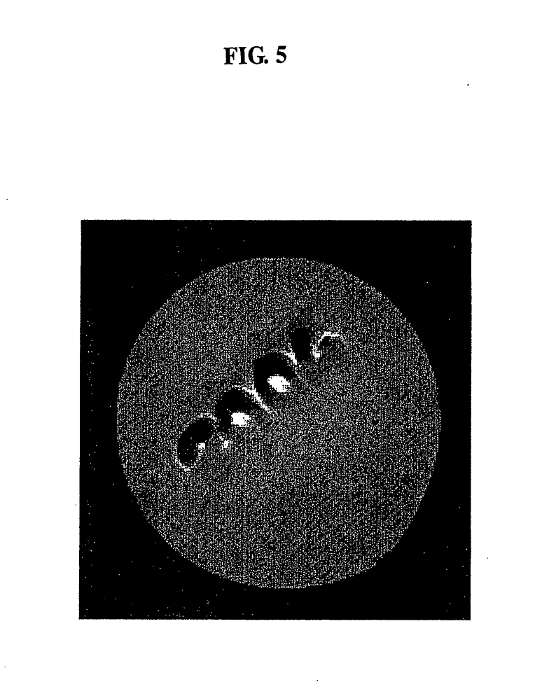

Further, the produced iron oxide-containing carbon nanohorn aggregate was dissolved in agar, and formed into a “2004” character-shaped gel. The gel was embedded in an agar gel containing no carbon nanohorn aggregates, and subjected to MRI. The obtained T1-MRI photograph image is shown in FIG. 4, and the T2-MRI photograph image is shown in FIG. 5.

As a result, it was confirmed that the iron oxide-containing carbon nanohorn aggregate is effective as a contrast agent.

### Example 3

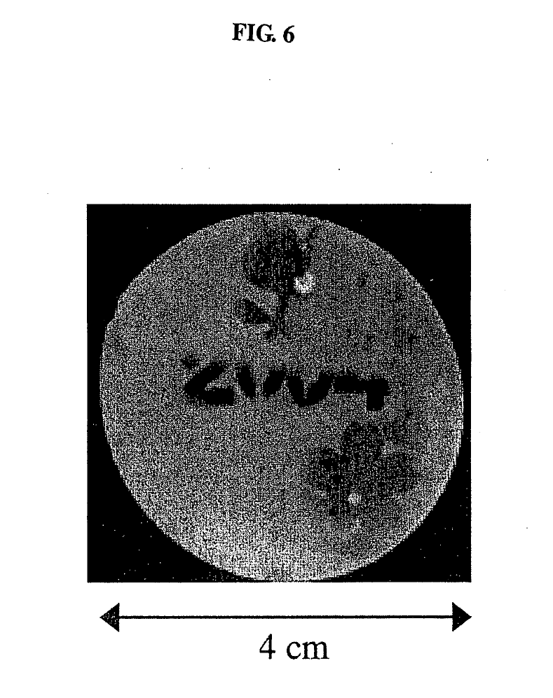

The carbon nanohorn aggregate produced in Example 1 was dissolved in an agar, and formed into a tulip-shaped gel, a cherry blossom-shaped gel, and a “2004” character-shaped gel. The gels were embedded in an agar gel containing no carbon nanohorn aggregates, and subjected to MRI. The T2 weighted photograph image thereof is shown in FIG. 6.

As a result, it was confirmed that the carbon nanohorn aggregate by itself is effective as a contrast agent.

### INDUSTRIAL APPLICABILITY

According to claim 1, the contrast agent has the advantages that it satisfies the requirement of low toxicity, enables microscopic diagnoses, and can be mass-produced more than fullerene contrast agents. Further, the contrast agent can envelop larger molecules.

According to claim 2, each of the carbon nanohorns in the carbon nanohorn aggregate has an opening at the side wall or tip, whereby the metal or the metal compound can be dispersed on the side wall or can be introduced through the opening into the nanohorn so that the metal as enveloped by the nanohorn. Thus, the contrast agent can be expected to have a higher contrast effect in addition to the above advantages.

According to claims 3 to 8, the contrast agent satisfies the requirement of low toxicity, enables microscopic diagnoses, can be mass-produced more easily than fullerene contrast agents, and further has a higher contrast effect. Further, in the case of incorporating the metal or the metal oxide in the carbon nanohorn aggregate, the carbon nanohorn aggregate surface can be chemically modified depending on the target living body, the range of available materials can be widened, the average particle diameter of the material can be controlled, and undesired aggregation can be prevented. Furthermore, in the case of using the oxide such as the Gd oxide or the Fe oxide, the carbon nanohorn aggregate is excellent in the stability, water resistance, acid resistance, and low toxicity.

The contrast agent of the invention can be used for detection of externally invisible, microscopic defects in materials and structures, etc.

1. A contrast agent characterized by comprising a carbon nanohom aggregate.

2. The contrast agent according to claim 1, wherein the carbon nanohorn aggregate comprises carbon nanohorns having an opening at the side wall or tip.

3. The contrast agent according to claim 1, wherein a metal M or a compound of the metal M is incorporated in each of the carbon nanohorns forming the carbon nanohorn aggregate, wherein M is at least one metal selected from the group consisting of paramagnetic metals, ferromagnetic metals, and superparamagnetic metals.

4. The contrast agent according to claim 1, wherein a metal M or a compound of the metal M is dispersed on a surface of each of the carbon nanohorns forming the carbon nanohorn aggregate, wherein M is at least one metal selected from the group consisting of paramagnetic metals, ferromagnetic metals, and superpara-magnetic metals.

5. The contrast agent according to claim 3, wherein the metal M or the compound of the metal M has an average particle diameter of 0.3 to 100 nm.

6. The contrast agent according to claim 3, wherein the compound of the metal M is a metal oxide.

7. The contrast agent according to claim 3, wherein the metal M is Gd or Fe.

8. The contrast agent according to claim 6, wherein the metal oxide is a Gd oxide or an Fe oxide.
